(19) United States
(12) Patent Application Publication
(10) Pub. No.: US 2025/0051303 A1
(43) Pub. Date: Feb. 13, 2025

(54) SUBSTITUTED OXOPYRIDINE DERIVATIVES FOR THE TREATMENT AND/OR PROPHYLAXIS OF THROMBOTIC OR THROMBOEMBOLIC DISORDERS AND/OR THROMBOTIC OR THROMBOEMBOLIC COMPLICATIONS

(71) Applicant: Bayer Aktiengesellschaft, Leverkusen (DE)

(72) Inventors: Stefan HEITMEIER, Wuelfrath (DE); Hardi MUNDL, Heidelberg (DE)

(21) Appl. No.: 18/925,716

(22) Filed: Oct. 24, 2024

Related U.S. Application Data
(62) Division of application No. 17/414,246, filed on Jun. 15, 2021.

(30) Foreign Application Priority Data
Dec. 17, 2018 (EP) 18213216.7

(51) Int. Cl. C07D 401/10; A61P 9/10

(52) U.S. Cl. CPC C07D 401/10; A61P 9/10

(57) ABSTRACT

The invention relates to the use of substituted oxopyridine derivatives for the treatment and/or prophylaxis of thrombotic or thromboembolic disorders and/or thrombotic or thromboembolie complications.

The invention relates to the use of substituted oxopyridine derivatives for the treatment and/or prophylaxis of thrombotic or thromboembolic disorders and/or thrombotic or thromboembolic complications.

Haemostasis is a protective mechanism of the organism, which helps to “seal” leaking damages in the blood vessel wall quickly and reliably. Thus, excessive loss of blood can often be avoided or kept to a minimum. After injury of a blood vessel, hemostasis is conducted mainly by activation and aggregation of platelets and activation the coagulation system, which consists of an enzymatic “waterfall” cascade leading one after another to the activation of the next coagulation factor until thrombin is formed, which leads to the generation of insoluble fibrin, which is an important part of the clot.

In the more recent past. the traditional theory of two separate starting points of the coagulation cascade (extrinsic and intrinsic path) has been modified owing to new findings: In these models, coagulation is initiated by binding of activated factor VIIa to tissue factor (TF). The resulting complex activates factor X. which in turn leads to generation of thrombin with subsequent production of fibrin and platelet activation (via PAR-1) as injury-scaling end products of hacmostasis. Compared to the subsequent amplification/propagation phase. the thrombin production rate in this first phase is low and as a result of the occurrence of TFPI as inhibitor of the TF-FVIIa-FX complex is limited in time. A central component of the transition from initiation to amplification of coagulation and thereby thrombus propagation is factor XIa: in positive feedback loops, thrombin activates not only factor V and factor VIII, but also factor XI to factor XIa, which in turn converts factor IX into factor IXa. which in turn in a factor IXa/factor VIIIa complex generates factor Xa and finally to large amounts of thrombin, resulting in strong thrombus growth and stabilization of the thrombus. This is supported by TAFIa and FXIIIa, which are activated by thrombin as well and lead to inhibition of clot lysis and further clot stabilisation.

In addition to the stimulation via tissue factor, the coagulation system can be activated particularly on negatively charged surfaces, which include not only surface structures of foreign cells (e.g. bacteria) but also artificial surfaces such as vascular prostheses, stents and extracoporeal circulation. On these surfaces, factor XII (FXII) is activated to factor XIIa, which subsequently activates factor XI to factor XIa. This leads to further activation of the coagulation cascade as described above. In addition, factor XIIa also activates bound plasma prokallikrein to plasma kallikrein (PK) which. in a potentiation loop. firstly leads to further factor XII activation, overall resulting in amplification of the initiation of this intrinsic part of the coagulation cascade.

Uncontrolled activation of the coagulation system or defective inhibition of the activation processes may lead to the formation of local thrombi or emboli in vessels (e.g. arteries, veins, lymph vessels) or in organ cavities (e.g. cardiac atrium). In addition, systemic hypercoagulability may lead to system-wide formation of microthrombi and finally to a consumption coagulopathy in the context of a disseminated intravasal coagulation. Thromboembolic complications may also occur in extracorporeal circulatory systems, such as haemodialysis, and also in vascular prostheses or prosthetic heart valves and stents.

In the course of many cardiovascular and metabolic disorders, increased tendency for coagulation and platelet activation occur owing to either systemic factors such as hyperlipidaemia, diabetes, inflammation, infection or smoking, or to changes in blood flow with stasis, for example in in diseased leg veins or in atrial fibrillation, or owing to pathological changes in vessel walls, for example endothelial dysfunctions or atherosclerosis. This unwanted and excessive activation of coagulation may, by formation of fibrin-and platelet-rich thrombi, lead to thromboembolic disorders and thrombotic complications with often life-threatening events. Inflammation processes may also be involved by triggering the coagulation system. On the other hand, thrombin is known to activate inflammatory pathways, as well.

Accordingly, thromboembolic disorders are still the most frequent cause of morbidity and mortality in most industrialized countries.

The anticoagulants known from the prior art, that is to say substances for inhibiting or preventing blood coagulation, have various disadvantages. Accordingly, in practice, efficient treatment methods or the prophylaxis of thrombotic/thromboembolic disorders is found to be difficult and unsatisfactory.

In the therapy and prophylaxis of thromboembolic disorders, use is made, firstly, of heparin which is administered parenterally or subcutaneously. Because of more favourable pharmacokinetic properties, preference is these days increasingly given to low-molecular-weight heparin; however, the known disadvantages described herein below encountered in heparin therapy cannot be avoided either in this manner. Thus, heparin is orally ineffective and has only a comparatively short half-life. In addition, there is a high risk of bleeding, there may in particular be cerebral haemorrhages and bleeding in the gastrointestinal tract, and there may be thrombopaenia, alopecia medicomentosa or osteoporosis. Low-molecular-weight heparins do have a lower probability of leading to the development of heparin-induced thrombocytopaenia; however, they can also only be administered subcutaneously. This also applies to fondaparinux, a synthetically produced selective factor Xa inhibitor having a long half-life.

A second class of anticoagulants are the vitamin K antagonists. These include, for example, 1,3-indanediones and in particular compounds such as warfarin, phenprocoumon, dicumarol and other coumarin derivatives which non-selectively inhibit the synthesis of various products of vitamin K-dependent coagulation factors in the liver. Owing to the mechanism of action, the onset of action is only very slow (latency to the onset of action 36 to 48 hours). The compounds can be administered orally; however, owing to the high risk of bleeding and the narrow therapeutic index complicated individual adjustment and monitoring of the patient are required. In addition, other side-effects such as gastrointestinal problems, hair loss and skin necroses have been described.

Today, approaches for Non-vitamin K dependent oral anticoagulantion (NOACs) are in clinical use, and have demonstrated their effectiveness in various studies. However, taking these medicaments can also lead to bleeding complications, particularly in predisposed patients.

Thus, for antithrombotic medicaments, the therapeutic window is of central importance: The interval between the therapeutically active dose for coagulation inhibition and the dose where bleeding may occur should be as large as possible so that maximum therapeutic activity is achieved at a minimum risk profile.

In various in vitro and in vivo models with, for example, antibodies as factor XIa inhibitors, but also in factor XIa knock-out animal models, the antithrombotic effect with small/no prolongation of bleeding time or extension of blood volume was confirmed. In clinical studies, elevated factor XIa concentrations were associated with an increased thrombotic event rate. In contrast, factor XI deficiency (haemophilia C) did not lead to spontaneous bleeding and was apparent only in the course of surgical operations and traumata, but did show protection with respect to certain thromboembolic events.

Furthermore, for many disorders the combination of antithrombotic and antiinflammtory principles may also be particularly attractive to prevent the mutual enhancement of coagulation and inflammation.

It is therefore an object of the present invention to provide novel compounds for the treatment and/or prophylaxis of cardiovascular disorders, in particular for the treatment and/or prophylaxis of disorders in the cerebrovascular arteries and/or and disorders of peripheral arteries, in humans and animals.

WO 2017/005725 describes substituted pyridin-2-ones and their use as factor XIa inhibitors.

The invention provides compounds of the formula

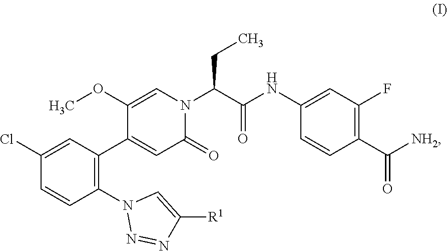

in which

R¹ represents trifluoromethyl or chlorine, and the salts thereof, the solvates thereof and the solvates of the salts thereof for use in the treatment and/or prophylaxis of disorders in the cerebrovascular arteries and/or disorders of peripheral arteries.

Compounds according to the invention are the compounds of the formula (I) and the salts, solvates and solvates of the salts thereof, and the salts, solvates and solvates of the salts thereof, to the extent that the compounds encompassed by formula (I) and specified hereinafter are not already salts, solvates and solvates of the salts.

The inventive compounds may, depending on their structure, exist in different stereoisomeric forms, i.e. in the form of configurational isomers or else, if appropriate, of conformational isomers (enantiomers and/or diastereomers, including those in the case of rotamers and atropisomers). The present invention therefore encompasses the enantiomers and diastereomers, and the respective mixtures thereof. The stereoisomerically uniform constituents can be isolated from such mixtures of enantiomers and/or diastereomers in a known manner; chromatography processes are preferably used for this, especially HPLC chromatography on an achiral or chiral phase.

If the compounds according to the invention can occur in tautomeric forms, the present invention encompasses all the tautomeric forms.

In the context of the present invention, the term “enantiomerically pure” is understood to mean that the compound in question with respect to the absolute configuration of the chiral centre is present in an enantiomeric excess of more than 95%, preferably more than 97%. The enantiomeric excess (ee value) is calculated in this case by evaluation of the corresponding HPLC chromatogram on a chiral phase with the aid of the formula below:

$$ee = [ {E}^{A} ( area \% ) - {E}^{B} ( area \% ) ] \times 100 \% / [ {E}^{A} ( area \% ) - {E}^{B} ( area \% ) ]$$

$$( {E}^{A} : enantiomer in excess , {E}^{B} : enantiomer in deficiency )$$

The present invention also encompasses all suitable isotopic variants of the compounds according to the invention. An isotopic variant of an inventive compound is understood here as meaning a compound in which at least one atom within the inventive compound has been exchanged for another atom of the same atomic number, but with a different atomic mass than the atomic mass which usually or predominantly occurs in nature. Examples of isotopes which can be incorporated into a compound according to the invention are those of hydrogen, carbon, nitrogen, oxygen, phosphorus, sulfur, fluorine, chlorine, bromine and iodine, such as 2H (deuterium), ³H (tritium), ¹³C, ¹⁴C, ¹⁵N, ¹⁷O, ¹⁸O, ³²p, ³³p, ³³S, ³⁴S, ³⁵S, ³⁶S, ¹⁸F, ³⁶C1, ⁸²Br, ¹²³I , ¹²⁴I , ¹²⁹I and ¹³¹I. Particular isotopic variants of a compound according to the invention, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the inventive compounds may therefore in some cases also constitute a preferred embodiment of the present invention. Isotopic variants of the compounds according to the invention can be prepared by the processes known to those skilled in the art, for example by the methods described further below and the procedures described in the working examples, by using corresponding isotopic modifications of the respective reagents and/or starting compounds.

Preferred salts in the context of the present invention are physiologically acceptable salts of the compounds according to the invention. However, the invention also encompasses salts which themselves are unsuitable for pharmaceutical applications but which can be used, for example, for the isolation or purification of the compounds according to the invention.

Physiologically acceptable salts of the compounds according to the invention include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

Solvates in the context of the invention are described as those forms of the inventive compounds which form a complex in the solid or liquid state by coordination with solvent molecules. Hydrates are a specific form of the solvates in which the coordination is with water.

The present invention additionally also encompasses prodrugs of the inventive compounds. The term “prodrugs” encompasses compounds which for their part may be biologically active or inactive but are converted during their residence time in the body into compounds according to the invention (for example by metabolism or hydrolysis).

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

The compound of the formula (I) in which R1 represents trifluoromethyl is the compound of the formula (Ia).

4-({(2S)-2-[4-{5-Chloro-2-[4-(trifluoromethyl)-1H-1,2,3-triazol-1-yl] phenyl}-5-methoxy-2-oxopyridin-1(2H)-yl]butanoyl}amino)-2-fluorobenzamide the compound of the formula

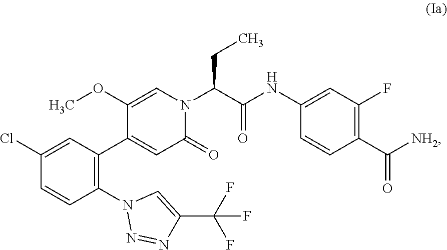 [SMILES: CC[C@@H](C(=O)Nc1ccc(C(N)=O)c(F)c1)n1cc(OC)c(-c2cc(Cl)ccc2-n2cc(C(F)(F)F)nn2)cc1=O]

or one of the salts thereof, solvates thereof or solvates of the salts thereof.

The compound of the formula (I) in which R¹ represents chlorine is the compound of the formula (Ib).

4-{[(2S)-2-{4-[5-Chloro-2-(4-chloro-1H-1,2,3-triazol-1-yl) phenyl]-5-methoxy-2-oxopyridin-1 (2H)-yl}butanoyl]amino}-2-fluorobenzamide the compound of the formula

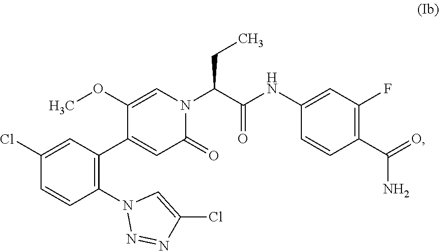 [SMILES: CC[C@@H](C(=O)Nc1ccc(C(N)=O)c(F)c1)n1cc(OC)c(-c2cc(Cl)ccc2-n2cc(Cl)nn2)cc1=O]

or one of the salts thereof, solvates thereof or solvates of the salts thereof.

Preference is given to the compound of the formula (Ia).

The invention provides 4- ({(2S)-2-[4-{5-chloro-2-[4-(trifluoromethyl)-1H-1,2,3-triazol-1-yl]phenyl}-5-methoxy-2-oxopyridin-1(2H)-yl]butanoyl}amino)-2-fluorobenzamide of the formula

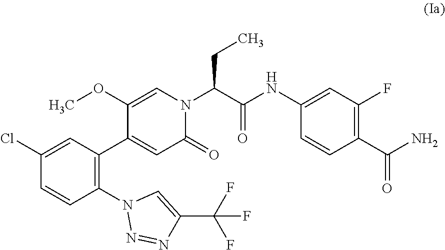 [SMILES: CC[C@@H](C(=O)Nc1ccc(C(N)=O)c(F)c1)n1cc(OC)c(-c2cc(Cl)ccc2-n2cc(C(F)(F)F)nn2)cc1=O]

or one of the salts thereof, solvates thereof or solvates of the salts thereof for use in the treatment and/or prophylaxis of disorders in the cerebrovascular arteries and/or disorders of peripheral arteries.

The invention provides 4-{[(2S)-2-{4-[5-chloro-2-(4-chloro-1H-1,2,3-triazol-1-yl)phenyl]-5-methoxy-2-oxopyridin-1(2H)-yl}butanoyl]amino}-2-fluorobenzamide of the formula

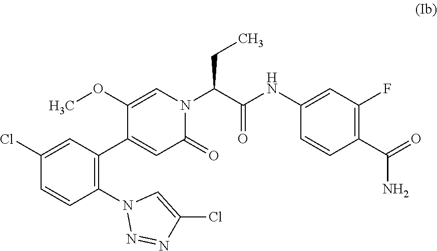 [SMILES: CC[C@@H](C(=O)Nc1ccc(C(N)=O)c(F)c1)n1cc(OC)c(-c2cc(Cl)ccc2-n2cc(Cl)nn2)cc1=O]

or one of the salts thereof, solvates thereof or solvates of the salts thereof for use in the treatment and/or prophylaxis of disorders in the cerebrovascular arteries and/or disorders of peripheral arteries.

Preference is given to the compounds of the formula (I) or one of the salts thereof, solvates thereof or solvates of the salts thereof for use in the treatment and/or prophylaxis of disorders in the cerebrovascular arteries, such as transitory ischaemic attacks (TIA), ischemic strokes including cardioembolic strokes, such as strokes due to atrial fibrillation, non-cardioembolic strokes, such as lacunar stroke, strokes due to large or small artery diseases, or strokes due to undetermined cause, cryptogenic strokes, embolic strokes, embolic strokes of undetermined source, or events of thrombotic and/or thromboembolic origin leading to stroke or TIA, and/or disorders of peripheral arteries, leading to peripheral artery disease, including peripheral artery occlusion, acute limb ischemia, amputation, reocclusions and restenoses after interventions such as angioplasty, stent implantation or surgery and bypass, and/or stent thrombosis.

Preference is also given to the compounds of the formula (I) or one of the salts thereof, solvates thereof or solvates of the salts thereof for use in the treatment and/or prophylaxis of disorders in the cerebrovascular arteries, such as transitory ischaemic attacks (TIA), ischemic strokes including cardioembolic strokes, such as strokes due to atrial fibrillation, non-cardioembolic strokes, such as lacunar stroke, strokes due to large or small artery diseases, or strokes due to undetermined cause, cryptogenic strokes, embolic strokes, embolic strokes of undetermined source, or events of thrombotic and/or thromboembolic origin leading to stroke or TIA.

Preference is also given to the compounds of the formula (I) or one of the salts thereof, solvates thereof or solvates of the salts thereof for use in the treatment and/or prophylaxis of disorders of peripheral arteries, leading to peripheral artery disease, including peripheral artery occlusion, acute limb ischemia, amputation, reocclusions and restenoses after interventions such as angioplasty, stent implantation or surgery and bypass, and/or stent thrombosis.

Preference is also given to the compounds of the formula (Ia) or one of the salts thereof, solvates thereof or solvates of the salts thereof for use in the treatment and/or prophylaxis of disorders in the cerebrovascular arteries, such as transitory ischaemic attacks (TIA), ischemic strokes including cardioembolic strokes, such as strokes due to atrial fibrillation, non-cardioembolie strokes, such as lacunar stroke, strokes due to large or small artery diseases, or strokes due to undetermined cause, cryptogenic strokes, embolic strokes, embolic strokes of undetermined source, or events of thrombotic and/or thromboembolic origin leading to stroke or TIA, and/or disorders of peripheral arteries, leading to peripheral artery disease, including peripheral artery occlusion, acute limb ischemia, amputation, reocclusions and restenoses after interventions such as angioplasty, stent implantation or surgery and bypass, and/or stent thrombosis.

Preference is also given to the compound of the formula (Ia) or one of the salts thereof, solvates thereof or solvates of the salts thereof for use in the treatment and/or prophylaxis of disorders in the cerebrovascular arteries, such as transitory ischaemic attacks (TIA), ischemic strokes including cardioembolic strokes, such as strokes due to atrial fibrillation, non-cardioembolic strokes, such as lacunar stroke, strokes due to large or small artery diseases, or strokes due to undetermined cause, cryptogenic strokes, embolic strokes, embolic strokes of undetermined source, or events of thrombotic and/or thromboembolic origin leading to stroke or TIA. Preference is also given to the compound of the formula (Ia) or one of the salts thereof, solvates thereof or solvates of the salts thereof for use in the treatment and/or prophylaxis of disorders of peripheral arteries, leading to peripheral artery disease, including peripheral artery occlusion, acute limb ischemia, amputation, reocclusions and restenoses after interventions such as angioplasty, stent implantation or surgery and bypass, and/or stent thrombosis.

The present invention further provides the compounds of the formula (I) or one of the salts thereof, solvates thereof or solvates of the salts thereof for use in the production of a medicament for the treatment and/or prophylaxis of disorders in the cerebrovascular arteries and/or disorders of peripheral arteries.

Preference is given to compounds of the formula (I) or one of the salts thereof, solvates thereof or solvates of the salts thereof for use in the production of a medicament for the treatment and/or prophylaxis of disorders in the cerebrovascular arteries, such as transitory ischaemic attacks (TIA), ischemic strokes including cardioembolic strokes, such as strokes due to atrial fibrillation, non-cardioembolic strokes, such as lacunar stroke, strokes due to large or small artery diseases, or strokes due to undetermined cause, cryptogenic strokes, embolic strokes, embolic strokes of undetermined source, or events of thrombotic and/or thromboembolic origin leading to stroke or TIA, and/or disorders of peripheral arteries, leading to peripheral artery disease, including peripheral artery occlusion, acute limb ischemia, amputation, reocclusions and restenoses after interventions such as angioplasty, stent implantation or surgery and bypass, and/or stent thrombosis.

The present invention further provides a method for the treatment and/or prophylaxis of disorders in the cerebrovascular arteries and/or disorders of peripheral arteries using a therapeutically effective amount of a the compounds of the formula (I) or one of the salts thereof, solvates thereof or solvates of the salts thereof.

Preference is given to a method for the treatment and/or prophylaxis of disorders in the cerebrovascular arteries, such as transitory ischaemic attacks (TIA), ischemic strokes including cardioembolic strokes, such as strokes due to atrial fibrillation, non-cardioembolic strokes, such as lacunar stroke, strokes due to large or small artery diseases, or strokes due to undetermined cause, cryptogenic strokes, embolic strokes, embolic strokes of undetermined source, or events of thrombotic and/or thromboembolic origin leading to stroke or TIA, and/or disorders of peripheral arteries, leading to peripheral artery disease, including peripheral artery occlusion, acute limb ischemia, amputation, reocclusions and restenoses after interventions such as angioplasty, stent implantation or surgery and bypass, and/or stent thrombosis, using a therapeutically effective amount of a the compounds of the formula (I) or one of the salts thereof, solvates thereof or solvates of the salts thereof.

The present invention further provides the compounds of the formula (I) or one of the salts thereof, solvates thereof or solvates of the salts thereof for use in a method for the treatment and/or prophylaxis of disorders in the cerebrovascular arteries and/or disorders of peripheral arteries, using a therapeutically effective amount of a compound of the formula (I).

Preference is given to the compounds of the formula (I) or one of the salts thereof, solvates thereof or solvates of the salts thereof for use in a method for the treatment and/or prophylaxis of disorders in the cerebrovascular arteries, such as transitory ischaemic attacks (TIA), ischemic strokes including cardioembolic strokes, such as strokes due to atrial fibrillation, non-cardioembolic strokes, such as lacunar stroke, strokes due to large or small artery diseases, or strokes due to undetermined cause, cryptogenic strokes, embolic strokes, embolic strokes of undetermined source, or events of thrombotic and/or thromboembolic origin leading to stroke or TIA, and/or disorders of peripheral arteries, leading to peripheral artery disease, including peripheral artery occlusion, acute limb ischemia, amputation, reocclusions and restenoses after interventions such as angioplasty, stent implantation or surgery and bypass, and/or stent thrombosis, using a therapeutically effective amount of a compound of the formula (I).

The present invention further provides medicaments comprising a compound of the formula (I) or one of the salts thereof, solvates thereof or solvates of the salts thereof for the treatment and/or prophylaxis of disorders in the cerebrovascular arteries and/or disorders of peripheral arteries.

Preference is given to medicaments comprising a compound of the formula (I) or one of the salts thereof, solvates thereof or solvates of the salts thereof for the treatment and/or prophylaxis of disorders in the cerebrovascular arteries, such as transitory ischaemic attacks (TIA), ischemic strokes including cardioembolic strokes, such as strokes due to atrial fibrillation, non-cardioembolic strokes, such as lacunar stroke, strokes due to large or small artery diseases, or strokes due to undetermined cause, cryptogenic strokes, embolic strokes, embolic strokes of undetermined source, or events of thrombotic and/or thromboembolic origin leading to stroke or TIA, and/or disorders of peripheral arteries, leading to peripheral artery disease, including peripheral artery occlusion, acute limb ischemia, amputation, reocclusions and restenoses after interventions such as angioplasty, stent implantation or surgery and bypass, and/or stent thrombosis.

The present invention further provides medicaments comprising a compound of the formula (I) or one of the salts thereof, solvates thereof or solvates of the salts thereof and one or more further active compounds for the treatment and/or prophylaxis of disorders in the cerebrovascular arteries and/or disorders of peripheral arteries.

Preference is given to medicaments comprising a compound of the formula (I) or one of the salts thereof, solvates thereof or solvates of the salts thereof and one or more further active compounds for the treatment and/or prophylaxis of disorders in the cerebrovascular arteries, such as transitory ischaemic attacks (TIA), ischemic strokes including cardioembolic strokes, such as strokes due to atrial fibrillation, non-cardioembolic strokes, such as lacunar stroke, strokes due to large or small artery diseases, or strokes due to undetermined cause, cryptogenic strokes, embolic strokes, embolic strokes of undetermined source, or events of thrombotic and/or thromboembolic origin leading to stroke or TIA, and/or disorders of peripheral arteries, leading to peripheral artery disease, including peripheral artery occlusion, acute limb ischemia, amputation, reocclusions and restenoses after interventions such as angioplasty, stent implantation or surgery and bypass, and/or stent thrombosis.

Preference is also given to the uses and medicaments for the compound of the formula (Ia).

The compounds according to the invention have an unforeseeable useful pharmacological activity spectrum and good pharmacokinetic properties. They are compounds that influence the proteolytic activity of the serine protease factor XIa (FXIa). The compounds according to the invention inhibit the enzymatic cleavage of FXIa-substrates, such as factor IX (FIX), which have essential roles in the activation of blood coagulation, in the aggregation of blood platelets via PAR-1 activation of the platelets, and in inflammatory processes, which particularly involve an increase in vascular permeability.

They are therefore suitable for use as medicaments for the treatment and/or prophylaxis of diseases in humans and animals.

The present invention further provides for the use of the compounds according to the invention for the treatment and/or prophylaxis of disorders, in particular vascular disorders, preferably thrombotic or thromboembolic disorders and/or thrombotic or thromboembolic complications.

Factor XIa (FXIa) is an important enzyme in the context of coagulation, which can be activated by both thrombin and factor XIIa (FXIIa), and is therefore involved in two essential processes of coagulation. It is a central component of the transition from initiation to amplification of the coagulation and propagation of the clot: in positive feedback loops, thrombin activates, in addition to factor V and factor VIII, also factor XI to factor XIa, whereby factor IX is converted into factor IXa, and, via the factor IXa/factor VIIIa complex generated in this manner, factor Xa and subsequently thrombin are formed, leading to strong thrombus growth and stabilization of the thrombus.

Moreover, factor XIa is an important component for the intrinsic initiation of coagulation: In addition to the stimulation via tissue factor (TF) in the extrinsic pathway, the coagulation system can be activated also particularly on negatively charged surfaces, which include not only surface structures of foreign cells (e.g. bacteria) but also artificial surfaces such as vascular prostheses, stents and parts of extracorporeal circulation systems. On these surfaces, factor XII (FXII) is activated to factor XIIa (FXIIa) which subsequently activates FXI to FXIa. This leads to further activation of the coagulation cascade as described above.

In contrast, thrombin generation triggered by TF/factor VIIa via factor X activation and finally thrombin formation, which represents the carly physiological reaction to vascular wall injuries, remains uninfluenced. This could explain why no prolongations of bleeding times were found in FXIa knockout mice, as in rabbits and other species, with administration of FXIa inhibitor. This low bleeding tendency caused by the substance is of great advantage for use in humans, particularly in patients with increased risk of bleeding.

Accordingly, the compounds according to the invention are suitable for the treatment and/or prophylaxis of disorders or complications which may arise from the formation of clots.

For the purpose of the present invention, the “thrombotic or thromboembolic disorders and/or thrombotic or thromboembolic complications” include disorders and complications, which occur in the arterial, the venous vascular system and the lymphatic system, which can be treated with the compounds according to the invention. This includes in particular disorders in the cerebrovascular arteries, such as transitory ischaemic attacks (TIA), ischemic strokes including cardioembolic strokes, such as strokes due to atrial fibrillation, non-cardioembolic strokes, such as lacunar stroke, strokes due to large or small artery diseases, or strokes due to undetermined cause, cryptogenic strokes, embolic strokes, embolic strokes of undetermined source, or events of thrombotic and/or thromboembolic origin leading to stroke or TIA, and disorders of peripheral arteries, leading to peripheral artery disease, including peripheral artery occlusion, acute limb ischemia, amputation, reocclusions and restenoses after interventions such as angioplasty, stent implantation or surgery and bypass, and/or stent thrombosis.

In addition, this includes thrombotic or thromboembolic disorders in particular in veins of the extremities, kidneys, mesenterium, liver, brain and eye, leading to pulmonary embolisms, venous thromboembolisms and/or venous thrombosis.

Moreover, the compounds according to the invention are suitable for the treatment and/or prophylaxis of disorders involving microclot formation or fibrin deposits in cerebral blood vessels or asymptomatic, covert strokes, which may lead to dementia disorders such as vascular dementia or Alzheimer's disease. Here, the clot may contribute to the disorder both via occlusions and by binding disease-relevant factors.

In addition, the compounds according to the invention may also be useful for the treatment of lung, liver and kidney fibrosis.

The present invention further provides for the use of the compounds according to the invention for the treatment and/or prophylaxis of disorders, especially the disorders mentioned above.

The present invention further provides for the use of the compounds according to the invention for production of a medicament for the treatment and/or prophylaxis of disorders, especially the disorders mentioned above. The present invention further provides a method for the treatment and/or prophylaxis of disorders, especially the disorders mentioned above, using a therapeutically effective amount of a compound according to the invention.

The present invention further provides the compounds according to the invention for use in a method for the treatment and/or prophylaxis of disorders, especially the disorders mentioned above, using a therapeutically effective amount of a compound according to the invention.

The present invention further provides medicaments comprising a compound according to the invention and one or more further active compounds.

The present invention further provides medicaments comprising a compound according to the invention and one or more further active compounds, in particular for the treatment and/or prophylaxis of the disorders mentioned above. Preferred examples of active compounds suitable for combinations include:
lipid-lowering substances, especially HMG-COA (3-hydroxy-3-methylglutaryl-coenzyme A) reductase inhibitors, for example lovastatin (Mevacor), simvastatin (Zocor), pravastatin (Pravachol), fluvastatin (Lescol) and atorvastatin (Lipitor);
coronary therapeutics/vasodilators, especially ACE (angiotensin converting enzyme) inhibitors, for example captopril, lisinopril, enalapril, ramipril, cilazapril, benazepril, fosinopril, quinapril and perindopril, or AII (angiotensin II) receptor antagonists, for example embusartan, losartan, valsartan, irbesartan, candesartan, eprosartan and temisartan, or β-adrenoceptor antagonists, for example carvedilol, alprenolol, bisoprolol, acebutolol, atenolol, betaxolol, carteolol, metoprolol, nadolol, penbutolol, pindolol, propanolol and timolol, or alpha-1-adrenoceptor antagonists, for example prazosine, bunazosine, doxazosine and terazosine, or diuretics, for example hydrochlorothiazide, furosemide, bumetanide, piretanide, torasemide, amiloride and dihydralazine, or calcium channel blockers, for example verapamil and diltiazem, or dihydropyridine derivatives, for example nifedipin (Adalat) and nitrendipine (Bayotensin), or nitro preparations, for example isosorbide 5-mononitrate, isosorbide dinitrate and glycerol trinitrate, or substances causing an increase in cyclic guanosine monophosphate (cGMP), for example stimulators of soluble guanylate cyclase, for example riociguat;
plasminogen activators (thrombolytics/fibrinolytics) and compounds which promote thrombolysis/fibrinolysis such as inhibitors of the plasminogen activator inhibitor (PAI inhibitors) or inhibitors of the thrombin-activated fibrinolysis inhibitor (TAFI inhibitors) such as, for example, tissue plasminogen activator (t-PA, for example Actilyse®), streptokinase, reteplase and urokinase or plasminogen-modulating substances causing increased formation of plasmin;
anticoagulatory substances (anticoagulants), for example heparin (UFH), low-molecular-weight heparins (LMW), for example tinzaparin, certoparin, parnaparin, nadroparin, ardeparin, enoxaparin, reviparin, dalteparin, danaparoid, semuloparin (AVE 5026), adomiparin (M118) and EP-42675/ORG42675;
direct thrombin inhibitors (DTI) such as, for example, Pradaxa (dabigatran), atecegatran (AZD-0837), DP-4088, SSR-182289A, argatroban, bivalirudin and tanogitran (BIBT-986 and prodrug BIBT-1011), hirudin;
direct factor Xa inhibitors, for example, rivaroxaban, apixaban, edoxaban (DU-176b), betrixaban (PRT-54021), R-1663, darexaban (YM-150), otamixaban (FXV-673/RPR-130673), letaxaban (TAK-442), razaxaban (DPC-906), DX-9065a, LY-517717, tanogitran (BIBT-986, prodrug: BIBT-1011), idraparinux and fondaparinux,
substances which inhibit the aggregation of platelets (platelet aggregation inhibitors, thrombocyte aggregation inhibitors), such as, for example, acetylsalicylic acid (such as, for example, aspirin), P2Y12 antagonists such as, for example, ticlopidine (Ticlid), clopidogrel (Plavix), prasugrel, ticagrelor, cangrelor, elinogrel, PAR-1 antagonists such as, for example, vorapaxar, PAR-4 antagonists, EP3 antagonists such as, for example, DG041;
platelet adhesion inhibitors such as GPVI and/or GPIb antagonists such as, for example, Revacept or caplacizumab;
fibrinogen receptor antagonists (glycoprotein-IIb/IIIa antagonists), for example abciximab, eptifibatide, tirofiban, lamifiban, lefradafiban and fradafiban;
recombinant human activated protein C such as, for example, Xigris or recombinant thrombomudulin;
and also antiarrhythmics.

“Combinations” for the purpose of the invention mean not only dosage forms which contain all the components (so-called fixed combinations) and combination packs which contain the components separate from one another, but also components which are administered simultaneously or sequentially, provided that they are used for prophylaxis and/or treatment of the same disease. It is likewise possible to combine two or more active ingredients with one another, meaning that they are thus each in two-component or multicomponent combinations.

The inventive compounds can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The inventive compounds can be administered in administration forms suitable for these administration routes.

Suitable administration forms for oral administration are those which function according to the prior art and deliver the inventive compounds rapidly and/or in modified fashion, and which contain the inventive compounds in crystalline and/or amorphized and/or dissolved form, for example tablets (uncoated or coated tablets, for example having enteric coatings or coatings which are insoluble or dissolve with a delay, which control the release of the compound according to the invention), tablets which disintegrate rapidly in the mouth, or films/wafers, films/lyophilisates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders. Preference is given to oral administration.

Suitable administration forms for the other administration routes are, for example, pharmaceutical forms for inhalation (including powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

The inventive compounds can be converted to the administration forms mentioned. This can be accomplished in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulfate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctants.

The present invention further provides medicaments comprising at least one inventive compound, preferably together with one or more inert nontoxic pharmaceutically suitable excipients, and the use thereof for the purposes mentioned above.

In the case of parenteral administration, it has generally been found to be advantageous to administer amounts 5 of about 5 to 250 mg every 24 hours to achieve effective results. In the case of oral administration, the amount is about 5 to 500 mg every 24 hours.

In spite of this, it may be necessary, if appropriate, to deviate from the amounts specified, specifically depending on body weight, administration route, individual behaviour towards the active ingredient, type of formulation, and time or interval of administration.

10 Unless stated otherwise, the percentages in the tests and examples which follow are percentages by weight; parts are parts by weight. Solvent ratios, dilution ratios and concentration data for the liquid/liquid solutions are based in each case on volume. “w/v” means “weight/volume”. For example, “10% w/v” means: 100 ml of solution or suspension comprise 10 g of substance.

### A) EXAMPLES

### Starting Materials

The syntheses of the starting materials are described in detail in WO 2017/005725.

### Synthesis Examples

### Example 1

4-({(2S)-2-[4-{5-Chloro-2-[4-(trifluoromethyl)-1H-1,2,3-triazol-1-yl] phenyl}-5-methoxy-2-oxopyridin-1(2H)-yl]butanoyl}amino)-2-fluorobenzamide of the formula (I)

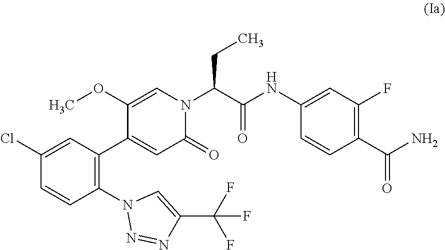 [SMILES: CC[C@@H](C(=O)Nc1ccc(C(N)=O)c(F)c1)n1cc(OC)c(-c2cc(Cl)ccc2-n2cc(C(F)(F)F)nn2)cc1=O]

or one of the salts thereof, solvates thereof or solvates of the salts thereof.

### Example 2

4-{[(2S)-2-{4-[5-Chloro-2-(4-chloro-1H-1,2,3-triazol-1-yl) phenyl]-5-methoxy-2-oxopyridin-1 (2H)-yl}butanoyl]amino}-2-fluorobenzamide of the formula (Ib)

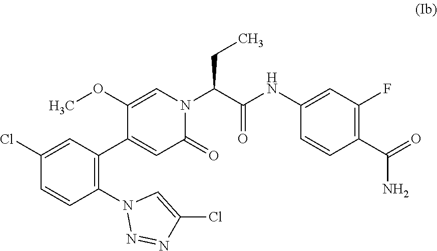 [SMILES: CC[C@@H](C(=O)Nc1ccc(C(N)=O)c(F)c1)n1cc(OC)c(-c2cc(Cl)ccc2-n2cc(Cl)nn2)cc1=O]

or one of the salts thereof, solvates thereof or solvates of the salts thereof.

The syntheses of the synthesis examples are described in detail in WO 2017/005725, see example 235 and 242.

### B) Assessment of Physiological Efficacy

The suitability of the compounds according to the invention for treating thromboembolic disorders can be demonstrated in the following assay systems:

a) Test descriptions (in vitro)

a. 1) Measurement of FXIa inhibition

The factor XIa inhibition of the substances according to the invention is determined using a biochemical test system which utilizes the reaction of a peptidic factor XIa substrate to determine the enzymatic activity of human factor XIa. Here, factor XIa cleaves from the peptidic factor XIa substrate the C-terminal aminomethylcoumarin (AMC), the fluorescence of which is measured. The determinations are carried out in microtitre plates.

Test substances are dissolved in dimethyl sulfoxide and serially diluted in dimethyl sulfoxide (3000 μM to 0.0078 μM; resulting final concentrations in the test: 50 μM to 0.00013 μM). In each case 1 μl of the diluted substance solutions is placed into the wells of white microtitre plates from Greiner (384 wells). 20 μl of assay buffer (50 mM of Tris/HCl pH 7.4; 100 mM of sodium chloride; 5 mM of calcium chloride; 0.1% of bovine serum albumin) and 20 μl of factor XIa from Kordia (0.45 nM in assay buffer) are then added successively. After 15 min of incubation, the enzyme reaction is started by addition of 20 ul of the factor XIa substrate Boc-Glu(OBzl)-Ala-Arg-AMC dissolved in assay buffer (10 μM in assay buffer) from Bachem, the mixture is incubated at room temperature (22° C.) for 30 min and fluorescence is then measured (excitation: 360 nm, emission: 460 nm). The measured emissions of the test batches with test substance are compared to those of control batches without test substance (only dimethyl sulfoxide instead of test substance in dimethyl sulfoxide), and IC50 values are calculated from the concentration/activity relationships. Activity data from this test are listed in Table A below (some as mean values from multiple independent individual determinations):

TABLE A

| Example No. | IC₅₀ [nM] |
|---|---|
| 1 | 0.92 |
| 2 | 0.89 |

a.2) Determination of the Selectivity

To demonstrate the selectivity of the substances with respect to FXIa inhibition, the test substances are examined for their potential to inhibit other human serine proteases, such as factor Xa, trypsin and plasmin. To determine the enzymatic activity of factor Xa (1.3 nmol/l from Kordia), trypsin (83 mU/ml from Sigma) and plasmin (0.1 ug/ml from Kordia), these enzymes are dissolved (50 mmol/l of Tris buffer [C,C,C-tris (hydroxymethyl) aminomethane], 100 mmol/l of NaCl, 0.1% BSA [bovine serum albumin], 5 mmol/l of calcium chloride, pH 7.4) and incubated for 15 min with test substance in various concentrations in dimethyl sulfoxide and also with dimethyl sulfoxide without test substance. The enzymatic reaction is then started by addition of the appropriate substrates (5 umol/l of Boc-Ile-Glu-Gly-Arg-AMC from Bachem for factor Xa and trypsin, 50 μmol/l of MeOSuc-Ala-Phe-Lys-AMC from Bachem for plasmin). After an incubation time of 30 min at 22° C., fluorescence is measured (excitation: 360 nm, emission: 460 nm). The measured emissions of the test mixtures with test substance are compared to the control mixtures without test substance (only dimethyl sulfoxide instead of test substance in dimethyl sulfoxide) and IC₅₀ values are calculated from the concentration/activity relationships.

a.3) Thrombin generation assay (thrombogram)

The effect of the test substances in the thrombin generation assay according to Hemker is determined in vitro in human plasma (Octaplas® from Octapharma).

In the thrombin generation assay according to Hemker, the activity of thrombin plasma is determined by measuring the fluorescent cleavage products of the substrate I-1140 (Z-Gly-Gly-Arg-AMC, Bachem). The reactions are carried out in the presence of varying concentrations of test substance or the corresponding solvent. To start the reaction, reagents from Thrombinoscope (30 pM to 0.1 pM recombinant tissue factor, 24 μM phospholipids in HEPES) are used. In addition, a thrombin calibrator from Thrombinoscope is used, of which the amidolytic activity is required for calculating the thrombin activity in a sample containing an unknown amount of thrombin. The test is carried out according to the manufacturer's instructions (Thrombinoscope BV): 4 μl of test substance or of the solvent, 76 μl of plasma and 20 μl of PPP reagent or thrombin calibrator are incubated at 37° C. for 5 min. After addition of 20 μl of 2.5 mM thrombin substrate in 20 mM Hepes, 60 mg/ml of BSA, 102 mM of calcium chloride, the thrombin generation is measured every 20 s over a period of 120 min. Measurement is carried out using a fluorometer (Fluoroskan Ascent) from Thermo Electron fitted with a 390/460 nm filter pair and a dispenser.

Using the Thrombinoscope software, the thrombogram is calculated and represented graphically. The following parameters are calculated: lag time, time to peak, peak, ETP (endogenous thrombin potential) and start tail.

a.4) Determination of anticoagulatory activity

The anticoagulatory activity of the test substances is determined in vitro in human plasma and rat plasma. Fresh whole blood is drawn directly into a mixing ratio of sodium citrate/blood of 1:9 using a 0.11 molar sodium citrate solution as receiver. Immediately after the blood has been drawn, it is mixed thoroughly and centrifuged at about 4000 g for 15 minutes. The supernatant is collected as (platelet-poor) plasma.

The prothrombin time (PT, synonyms: thromboplastin time, quick test) is determined in the presence of varying concentrations of test substance or the corresponding solvent using a commercial test kit (Neoplastin® from Bochringer Mannheim or Hemoliance® RecombiPlastin from Instrumentation Laboratory). The test compounds are incubated with plasma at 37° C. for 3 minutes. Coagulation is then started by addition of thromboplastin, and the timepoint, at which clotting of the sample occurs is determined. The concentration of test substance which effects a doubling of the prothrombin time is determined.

The activated partial thromboplastin time (APTT) is determined in the presence of varying concentrations of test substance or the corresponding solvent using a commercial test kit (PTT reagent from Roche). The test compounds are incubated with the plasma and the PTT reagent (cephalin, kaolin) at 37° C. for 3 minutes. Coagulation is then started by addition of 25 mM calcium chloride, and the time when coagulation occurs is determined. The concentration of test substance which leads to an extension by 50% or a doubling of the APTT is determined.

a.5) Determination of the plasma kallikrein activity

To determine the plasma kallikrein inhibition of the substances according to the invention, a biochemical test system is used which utilizes the reaction of a peptidic plasma kallikrein substrate to determine the enzymatic activity of human plasma kallikrein. Here, plasma kallikrein cleaves from the peptidic plasma kallikrein substrate the C-terminal aminomethylcoumarin (AMC), the fluorescence of which is measured. The determinations are carried out in microtitre plates.

Test substances are dissolved in dimethyl sulfoxide and serially diluted in dimethyl sulfoxide (3000 μM to 0.0078 μM; resulting final concentrations in the test: 50 uM to 0.00013 pM). In each case 1 μl of the diluted substance solutions is placed into the wells of white microtitre plates from Greiner (384 wells). 20 ul of assay buffer (50 mM Tris/HCI pH 7.4; 100 mM sodium chloride solution; 5 mM of calcium chloride solution; 0.1% of bovine serum albumin) and 20 μl of plasma kallikrein from Kordia (0.6 nM in assay buffer) are then added successively. After 15 min of incubation, the enzyme reaction is started by addition of 20 μl of the substrate H-Pro-Phe-Arg-AMC dissolved in assay buffer (10 UM in assay buffer) from Bachem, the mixture is incubated at room temperature (22° C.) for 30 min and fluorescence is then measured (excitation: 360 nm, emission: 460 nm). The measured emissions of the test batches with test substance are compared to those of control batches without test substance (only dimethyl sulfoxide instead of test substance in dimethyl sulfoxide), and IC₅₀ values are calculated from the concentration/activity relationships. Activity data from this test are listed in Table B below (some as mean values from multiple independent individual determinations):

TABLE B

| Example No. | IC₅₀ [nM] |
|---|---|
| 1 | 5.7 |
| 2 | 5.4 |

b) Determination of antithrombotic activity (in vivo) b.1) Arterial thrombosis model (iron (II) chloride-induced thrombosis) in combination with ear bleeding time in rabbits

The antithrombotic activity of the FXIa inhibitors is tested in an arterial thrombosis model. Thrombus formation is triggered here by causing chemical injury to a region in the carotid artery in rabbits. Simultaneously, the car bleeding time is determined.

Male rabbits (Crl: KBL (NZW) BR. Charles River) receiving a normal diet and having a body weight of 2.2-2.5 kg are anaesthetized by intramuscular administration of xylazine and ketamine (Rompun, Bayer. 5 mg/kg and Ketavet, Pharmacia & Upjohn GmbH, 40 mg/kg body weight). Anaesthesia is maintained by intravenous administration of the same preparations (continuous infusion) via the right auricular vein.

The right carotid artery is exposed and the vessel injury is caused by wrapping a piece of filter paper (10 mm x mm) on a Parafilm® strip (25 mm×12 mm) around the carotid artery without disturbing the blood flow.

10 The filter paper contains 100 μL of a 13% strength solution of iron (II) chloride (Sigma) in water. After 5 min. the filter paper is removed and the vessel is rinsed twice with aqueous 0.9% strength sodium chloride solution. 30 min after the injury the injured region of the carotid artery is extracted surgically and any thrombotic material is removed and weighed.

The test substances are administered either intravenously to the anaesthetized animals via the femoral vein or orally to the awake animals via gavage, in each case 5 min and 2 h. respectively, before the injury.

Ear bleeding time is determined 2 min after injury to the carotid artery. To this end. the left ear is shaved and a defined 3-mm-long incision (blade Art. Number 10-150-10, Martin, Tuttlingen, Germany) is made parallel to the longitudinal axis of the car. Care is taken not to damage any visible vessels. Any blood that extravasates is taken up in 15 second intervals using accurately weighed filter paper pieces, without touching the wound directly. Bleeding time is calculated as the time from making the incision to the point in time when no more blood can be detected on the filter paper. The volume of the extravasated blood is calculated after weighing of the filter paper pieces.

c) Determination of permeability (Caco assay)

The Caco cells (obtained from the Deutsche Sammlung für Mikroorganismen and Zellkulturen, DSMZ) are cultivated in 24-well Transwell plates for 15 or 16 days. The test is carried out using a Hamilton robot. The density of the cell monolayers is ensured by measuring the Lucifer yellow permeability. The test compounds are dissolved in DMSO and then diluted with assay buffer to a concentration of 2 μM (final DMSO concentration 1%). The permeability is examined in both directions by addition of the substance solutions to the apical or basolateral compartment. The covered plates are incubated at 37° C. for 2 hours. The concentrations in the two compartments are determined by LC-MS/MS and the Papp values are calculated according to Artursson and Karlsson (PMID: 1673839).

d) Determination of pharmacokinetic parameters following intravenous administration

To examine the pharmacokinetic properties of a test substance, the respective test substances are administered to animals as a bolus injection, infusion or via oral administration. In the case of rats, the preferred formulation for intravenous administration of the test substances is plasma/dimethyl sulfoxide in a ratio of 99:1. The infusion solution of the test substance in the case of dogs and monkeys consists of polyethylene glycol/ethanol/water in a ratio of 50/10/40. Formulations for oral administration can be polyethylene glycol/ethanol/water or solutol/ethanol/water in a ratio of 50/10/40, or other formulations as appropriate (e.g. water, tylose, self-emulsifying drug dispering systems, etc.). The administration volume for rats is 2-10 ml/kg, for dogs and monkeys 0.5-5 ml/kg.

Blood samples are removed from the test animals into sodium EDTA (or other anticoagulant)-containing tubes: in the case of bolus administration, blood samples are usually taken at 0.033, 0.083, 0.167, 0.25, 0.283, 0.333, 0.5, 0.75, 1, 2, 3, 5, 7, 24 hours after administration of the test substance. In the case of infusions, blood samples are usually taken at 0.083, 0.167, 0.25, 0.283, 0.333, 0.5, 0.75, 1, 2, 3, 5, 7, 24 hours after administration of the test substance. In the case of oral administration, blood samples are usually taken at 0.083, 0.25, 0.5, 0.75, 1, 2, 3. 5. 7. 24 hours after administration of the test substance. Other time points might be chosen as appropriate.

After removal, the blood samples are centrifuged at 1280 g for 10 minutes. The supernatant (plasma) is taken off and either directly processed further or frozen for later sample preparation. For sample preparation, 50 ul of plasma are mixed with 250 ul of acetonitrile (the precipitating agent acetonitrile also contains the internal standard ISTD for later analytical determination) and then allowed to stand at room temperature for 5 minutes. The mixture is then centrifuged at 16 000 g for 3 minutes. The supernatant is taken off, and 500 ul of a buffer suitable for the mobile phase are added. The samples are then examined by LC-MS/MS analysis (e.g. liquid chromatography using a Gemini 5 μM C18 110A 50 mm×3 mm (or 150 mm×3 mm) column from Phenomenex; by mass spectrometry using an API 5500 or API 6500; SCIEX, Canada) to determine the concentration of the test substance in the individual samples.

In addition to the the plasma concentrations, the concentration ratio whole blood to plasma for the test substance in question is determined. To this end, the test substance is incubated at a certain concentration in whole blood for 20 minutes. The samples are then processed as described above to determine the concentration of the test substance in the plasma. The concentration set divided by the concentration measured in the plasma gives the parameter Cb/Cp.

The pharmacokinetic parameters are calculated by non-compartmental analysis (NCA). The algorithms for calculating the parameters are defined in an internal process description and are based on rules published in general textbooks of pharmacokinetics.

The primary pharmacokinetic parameters clearance (CL) and distribution volume (Vss) are calculated as follows:

| Parameter | Formula |
|---|---|
| CLplasma (plasma clearance) | CLplasma = dose/AUC (AUC = area under the curve) |
| CLblood (blood clearance) | CLblood = CLplasma/(Cb/Cp) |
| Vss | Vss = CLplasma * MRTiv |
| MRTiv | MRTiv = AUMC/AUC |
| AUMC | AUMC = AUMC(0-tₗₐₛₜ) + tₗₐₛₜ*C_{last, calculated}/λ_{Z} + C_{last, calculated}/λ_{Z}² |
| λ_{Z} | Rate constant for the terminal phase; calculated from the |
| | logarithmic-linear regression of unweighted data from the |
| | terminal phase with data points above the detection limit |
| AUC | AUC = AUC(0-tlast) + C_{last, calculated}/λ_{Z} |
| AUCnorm | AUC divided by dose (mg) per kg body weight |

C) Working examples of pharmaceutical compositions

The substances according to the invention can be converted to pharmaceutical preparations as follows:

Tablet:

Composition:

100 mg of the compound of Example 1, 50 mg of lactose (monohydrate), 50 mg of maize starch, 10 mg of polyvinylpyrrolidone (PVP 25) (from BASF, Germany) and 2 mg of magnesium stearate.

Tablet weight 212 mg. Diameter 8 mm, radius of curvature 12 mm.

Production:

The mixture of the compound of Example 1, lactose and starch is granulated with a 5% strength solution (m/m) of the PVP in water. After drying, the granules are mixed with the magnesium stearate for 5 min. This mixture is compressed in a conventional tabletting press (see above for format of the tablet).

Oral suspension:

Composition:

1000 mg of the compound of Example 1, 1000 mg of ethanol (96%), 400 mg of Rhodigel (xanthan gum) (from FMC, USA) and 99 g of water.

10 ml of oral suspension correspond to a single dose of 100 mg of the compound of the invention.

Production:

The Rhodigel is suspended in ethanol, and the compound of Example 1 is added to the suspension. The water is added while stirring. The mixture is stirred for about 6 h until swelling of the Rhodigel is complete.

1-6. (canceled)

7. Compound of the formula (I) or one of the salts thereof, solvates thereof or solvates of the salts thereof according to claim 1 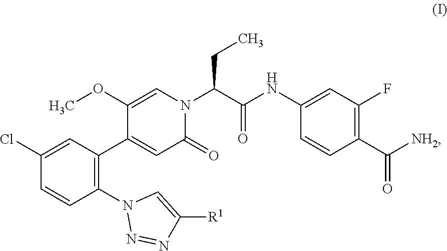
in which
represents trifluoromethyl or chlorine,
for use in a method for the treatment and/or prophylaxis of disorders in the cerebrovascular arteries and/or disorders of peripheral arteries, administering a therapeutically effective amount of a compound of the formula (I) to a patient in need thereof.

8. Compound of the formula (I) or one of the salts thereof, solvates thereof or solvates of the salts thereof 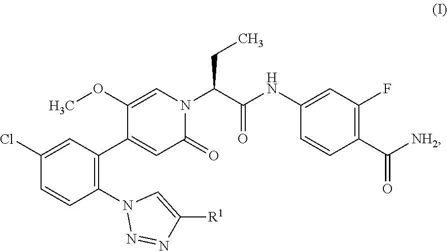
in which
R¹ represents trifluoromethyl or chlorine,
for use in a method for the treatment and/or prophylaxis of disorders in the cerebrovascular arteries, such as transitory ischaemic attacks (TIA), ischemic strokes including cardioembolic strokes, such as strokes due to atrial fibrillation, non-cardioembolic strokes, such as lacunar stroke, strokes due to large or small artery diseases, or strokes due to undetermined cause, cryptogenic strokes, embolic strokes, embolic strokes of undetermined source, or events of thrombotic and/or thromboembolic origin leading to stroke or TIA, and/or disorders of peripheral arteries, leading to peripheral artery disease, including peripheral artery occlusion, acute limb ischemia, amputation, reocclusions and restenoses after interventions such as angioplasty, stent implantation or surgery and bypass, and/or stent thrombosis, administering a therapeutically effective amount of a compound of the formula (I) to a patient in need thereof.

9. Medicament comprising a compound of the formula (I) or one of the salts thereof, solvates thereof or solvates of the salts thereof 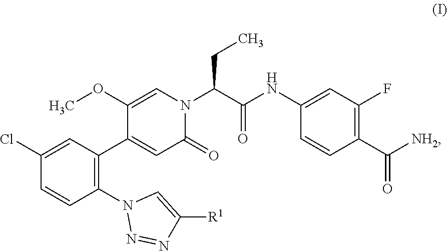
in which
R¹ represents trifluoromethyl or chlorine,
for the treatment and/or prophylaxis of disorders in the cerebrovascular arteries and/or disorders of peripheral arteries.

10. Medicament comprising a compound of the formula (I) or one of the salts thereof, solvates thereof or solvates of the salts thereof 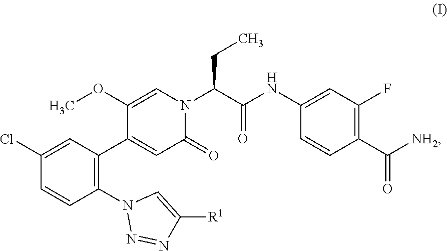
in which
R¹ represents trifluoromethyl or chlorine,
for the treatment and/or prophylaxis of disorders in the cerebrovascular arteries, such as transitory ischaemic attacks (TIA), ischemic strokes including cardioembolic strokes, such as strokes due to atrial fibrillation, non-cardioembolic strokes, such as lacunar stroke, strokes due to large or small artery diseases, or strokes due to undetermined cause, cryptogenic strokes, embolic strokes, embolic strokes of undetermined source, or events of thrombotic and/or thromboembolic origin leading to stroke or TIA, and/or disorders of peripheral arteries, leading to peripheral artery disease, including peripheral artery occlusion, acute limb ischemia, amputation, reocclusions and restenoses after interventions such as angioplasty, stent implantation or surgery and bypass, and/or stent thrombosis.
